(19) United States
(12) Patent Application Publication
(10) Pub. No.: US 2010/0256464 A1
(43) Pub. Date: Oct. 7, 2010

(54) DEVICE FOR DETERMINING RISK OF DEVELOPING BREAST CANCER AND METHOD THEREOF

(75) Inventors: Susan Love, Pacific Palisades, CA (US); Jan Wan, San Marino, CA (US)

Correspondence Address: KNOBBE MARTENS OLSON & BEAR LLP, 2040 MAIN STREET, FOURTEENTH FLOOR, IRVINE, CA, 92614, US

(73) Assignee: Dr. Susan Love Research Foundation, Santa Monica, CA (US)

(21) Appl. No.: 12/600,245

(22) PCT Filed: May 13, 2008
(86) PCT No.: PCT/US08/63545

§ 371 Date: Nov. 13, 2009

Related U.S. Application Data
(60) Provisional application No. 60/917,892, filed on May 14, 2007.

(51) Int. Cl. A61B 10/00

(52) U.S. Cl. 600/309

(57) ABSTRACT

A cost-effective and sensitive apparatus and method for detecting individuals at risk for developing or having breast cancer is provided. More particularly, an apparatus and method for detecting markers associated with breast cancer risk is provided in one embodiment. The apparatus and method use nipple aspirate fluid obtained from breast ducts. This nipple aspirate fluid is applied to a marker panel that tests for the presence of specific markers associated with breast cancer risk. The apparatus and method provide a positive response based upon the detection of at least one marker associated with an increased risk for developing or having breast cancer.

### RELATED APPLICATION

This patent application claims priority to Provisional Patent Application No. 60/917,892 filed May 14, 2007, the entirety of which is incorporated herein by reference.

### BACKGROUND OF THE INVENTION

1. Field of the Invention

Embodiments of the invention relate generally to systems and methods for pre-screening individuals who may be at risk for breast cancer, so that such individuals can be referred for screening.

2. Description of the Related Art

Breast cancer is a significant health problem in the industrialized western world, where it is the most common form of cancer among women in North America and almost all of Europe. It is estimated that each year the disease is diagnosed in over one million women worldwide and is the cause of death in over 400,000 women. The incidence of the disease is increasing in both industrialized and developing countries.

Although mammography screening has had some success in identifying tumors in asymptomatic women in the United States and Europe, it has limitations that make it less useful in the rest of the world. It is expensive to apply and requires trained technicians using high-tech equipment to achieve images that must then be interpreted by specialized radiologists. Mobile vans that take digital mammograms that can be read centrally have been developed in an attempt to overcome this problem, but these still require the use of scarce resources. The World Health Organization states that “mammography screening is an expensive test that requires great care and expertise both to perform and in the interpretation of results. It is therefore currently not a viable option for many countries.”

Further, mammography is most accurate in postmenopausal women, because their breasts tend to be less dense than those of women of reproductive age. In the United States and Europe, breast cancer is more common in postmenopausal Caucasian women, making mammographic screening a reasonable approach. For reasons not yet fully understood, in countries outside the United States and Europe, including many parts of the developing world, the majority of breast cancer cases occur in premenopausal women. In China, for example, the mean age of diagnosis is 48. For this population, mammography is not only expensive, but also not particularly sensitive.

Breast self-exam, whereby an individual massages the breast in search of tumors or other tissue abnormalities, might seem to be an answer to screening premenopausal women, since it is indeed cost-effective. However, a large well-designed study conducted in China to explore the utility of breast self-exam found that women who conducted monthly breast self-exams were no more likely to find their tumors at an earlier stage and no less likely to die of breast cancer than were women who did not perform regular breast self-exam. Even clinical breast exam has limited evidence of effectiveness in reducing breast cancer mortality. The World Health Organization states that: “Given the present level of evidence, the national cancer control programme should not recommend screening by breast self exam and physical examinations of the breast.”

Immunoassays, biological tests that measure the concentration of a substance in a biological liquid, use the specific binding of an antibody to an antigen as a means to determine the presence of an antibody or antigen. Thus, the marker detector antibody or antigen binds the antibody or antigen that is being tested for—the marker. For example, if it is desired to determine whether biological liquid contains a specific antibody, the marker, a corresponding antigen that binds specifically to that marker can be used to test for the marker's presence in the biological liquid. Thanks to modern developments in immunoassay techniques, markers can be detected from considerably smaller samples than were required for previous chemical assay methods. In addition to allowing for smaller samples, developments in immunoassay technology have also allowed for lower-cost options in immunoassay testing.

One example of a modern product reaping the benefits of these developments is the home pregnancy test. Home pregnancy tests typically operate by testing for the presence of one or more pregnancy markers, such as hCG, in the urine. The typical home pregnancy test contains an immunoassay strip, formed by compressing non-woven fibers into a narrow strip and thereafter coating the fiber strip with antibodies that react to antigen pregnancy markers, as well as an absorbent pad for collecting the urine sample that is applied the immunoassay strip.

### SUMMARY

There exists a need for a cost-effective and sensitive apparatus and method for detecting individuals at risk for developing or having breast cancer, but who do not necessarily have breast cancer. There remains a need for an efficient system that is adapted for large scale screening of individuals to determine which individuals should seek further testing for breast cancer. Thus, in one embodiment, the system is a pre-screening test in that it determines which individuals should be screened further.

In one embodiment, the invention comprises a simple, inexpensive, sensitive screening test for breast cancer risk. According to one embodiment, this type of test would not be used to detect breast cancer, but would be used to identify women who were at risk of having or developing breast cancer. The women who test positive could then be further screened for a malignancy by imaging or one of the cytology techniques being developed, or be considered for preventative measures. Because it would be used to determine which women needed further noninvasive workup and not as a final determinant of the presence of breast cancer, a test that screens for breast cancer risk would be useful even if it had a higher false positive rate than would be acceptable for a breast cancer detection screening test. In another embodiment, a screening test would be used to identify individuals who have developed breast cancer.

In one embodiment, the invention comprises a pre-screening system for detecting individuals at risk for breast cancer (or at higher risk for breast cancer than the average population risk). In one embodiment, the system comprises a capture element for capturing ductal fluid from a breast duct and at least one marker panel (or other support system). The marker panel comprises at least one marker detector. The marker detector is adapted to detect one or more markers associated with breast cancer risk. The system indicates a positive response upon detection of at least one marker. Adhesive may be used to secure the system to the patient. In one embodiment, this system is adapted to assay small quantities of ductal fluid, such as 1-5 μl, 5-10 μl, 10-15 μl, 15-20 μl, 20-30 μl, 30-50 μl, and 50-500 μl.

In one embodiment, the invention comprises a method for pre-screening individuals at risk for having or developing breast cancer. In one embodiment the method comprises capturing ductal fluid from a breast duct and contacting said ductal fluid to at least one marker detector. The marker detector is adapted to detect markers associated with breast cancer risk. The method further comprises indicating a positive response upon detection of at least one marker. Optionally, the method further comprises indicating the presence of ductal fluid.

In one embodiment, a positive response includes a color change. A control marker may be used in some embodiments as a quality control measure.

In one embodiment, the capture element comprises a well or absorption portion. In one embodiment, the capture element captures fluid from the duct. In another embodiment, the capture element facilitates drawing or obtaining the fluid. In one embodiment, the system comprises a portion to carry or facilitate transport of the captured fluid to the marker detectors.

In one embodiment, the marker detector comprises an antibody. In one embodiment, the marker detector is adapted to detect a marker selected from the group consisting of one or more of the following markers: bFGF, Apolipoprotein D, Mammaglobin, PSA, Vitamin D binding protein, GCDFP15, a-Lactoalbumin, Cathepsin D, Annexin I/II/V, Free Her-2, Alpha-fetoprotein, C Reactive Protein, Carcinoembryonic antigen and urokinase, and their respective receptors. In one embodiment, the markers comprise PSA and Alpha-fetoprotein, together or in combination with any one of the markers identified herein. In another embodiment, the markers consist only of or only essentially of PSA and Alpha-fetoprotein. In one embodiment, the markers comprise EGF, Apolipoprotein D and urokinase, together or in combination with PSA and Alpha-fetoprotein.

In one embodiment, the system comprises a detector to detect the presence of ductal fluid. The volume of fluid can also be detected in alternative embodiments. An indicator may be used to indicate either the presence or volume of ductal fluid. The indicator can be, for example, a color change.

In one embodiment, the system comprises a first antibody conjugated with gold to form a conjugate and deposited on the panel and a second antibody immobilized onto a nitrocellulose membrane (or on another portion of the panel). The ductal fluid is adapted to dissolve the conjugate to form a liquid mixture, which is adapted to move onto the nitrocellulose membrane (e.g., by capillary action) and contact the second antibody. A sandwich of the conjugate and the second antibody will form if a marker is present in the ductal fluid, and a color will form if the sandwich is formed.

In one embodiment, the invention comprises a kit. In one embodiment, the kit comprises one of the Breast Fluid Test systems described herein and instructions for use. In another embodiment, the kit comprises an aspirator or other device for facilitating capture of ductal fluid.

Certain embodiments of the present invention are particularly advantageous because the assay can be used to determine individual risk, as compared to population risk. The Gail Index is an epidemiological based model which is currently being applied to determine risk in the United States. Although it works very well at predicting populations at risk, it is less accurate in predicting individual risk and is directed to the US population. It also is not very sensitive, underestimating women with BRCA1 or 2 and estrogen receptor negative tumors.

Some embodiments of the present invention are particularly advantageous because they provide a cost-effective “low-tech” breast cancer risk assessment pre-screening tool that can be used at home. Thus, although the breast cancer risk assessment test could be used in hospitals and clinics, a woman would not need access to a hospital or clinic to use the test. By contrast, diagnostic techniques currently being employed require expensive imaging hospital equipment to image cancers and pre-cancers. Although mammographic density is beginning to be used in the United States to identify risk for breast cancer, mammographic density still requires expensive technology that is not available in most of the world.

Preferred embodiments of the present invention are expected to have a direct and immediate impact on the health of women in this country and around the world.

In one preferred embodiment, a system for pre-screening individuals at risk for breast cancer is provided, the system being comprised of a well for capturing ductal fluid from a breast duct and at least one panel (or other support system) comprising at least one marker detector. The marker detector comprises an antibody or antigen which detects one or more markers selected from the following group: bFGF, Apolipoprotein D, mammaglobin, PSA, VDR, GCDFP15, a-lactoalbumin, Cathepsin D, Annexin I/II/V, Free Her-2, Alpha-fetoprotein, C Reactive Protein, Carcinoembryonic antigen and urokinase, and their respective receptors The marker detector is adapted to bind to said markers which are associated with breast cancer risk. The system indicates a positive response upon detection of at least one of said markers.

In another embodiment, the system described above includes an adhesive for securing the system to a patient.

In another embodiment, the system described above comprises five marker panels, wherein each panel comprises a marker detector. In another embodiment, the system described above comprises a single panel comprising one or more marker detectors.

In another embodiment, a positive response is indicated when at least one marker is detected. In another embodiment a positive response is indicated when all markers are detected. In another embodiment, a positive response is indicated by a color change.

In another embodiment, the well of the system described above is adapted to draw fluid from the breast duct. In another embodiment the well is adapted to capture fluid from the breast duct. In another embodiment, the system described above comprises a portion to facilitate transport of the captured fluid to the marker detectors.

In another embodiment, the system described above further comprises a detector for detecting the presence of ductal fluid. In another embodiment, the system further comprises a detector to detect the volume of ductal fluid captured. In yet another embodiment the system further comprises an indicator to indicate the presence of ductal fluid or a certain volume of ductal fluid. In one embodiment, the indicators described comprise a color change.

In one embodiment a kit comprises instruction for use of the system. In another embodiment a kit comprises an aspirator for facilitating the capture of ductal fluid.

In another preferred embodiment, a method for pre-screening individuals at risk for breast cancer is provided where ductal fluid is captured from a breast duct. The ductal fluid is then contacted to at least one marker detector which is adapted to detect markers associated with breast cancer risk and indicate a positive response upon the detection of at least one of these markers. In another embodiment, the method described further comprises detecting the presence of ductal fluid.

In another preferred embodiment, a system for identifying individuals at risk for breast cancer is provided where the system comprises a well for capturing ductal fluid from a breast duct and at least one panel comprised of at least one marker detector adapted to detect markers associated with breast cancer risk. The system indicates a positive response upon detection of at least one marker.

### BRIEF DESCRIPTION OF THE DRAWINGS

For a better understanding of the invention, and to show how it may be carried into effect, reference will be made, but way of example, to the accompanying drawings, in which:

FIG. 1A shows a suction cup and collection device embodiment where the suction cup includes a collection device for collecting ductal fluid.

FIG. 1B shows an alternative suction cup and collection device embodiment where the collection device is interposed between the suction cup and the source of suction.

FIG. 2A shows a simple diagram for a two marker test strip.

FIG. 2B shows a simple diagram for a five marker test strip.

FIG. 3 shows a star-shaped embodiment of the Breast Fluid Test comprising a central well and five test strips leading away from the central well.

FIGS. 4A and 4B show two alternative bandage embodiment of the Breast Fluid Test comprising a test strip centered on an adhesive backing.

FIG. 5 shows a dipstick Breast Fluid Test comprising a fluid collection device and in which a test strip is connected to the fluid collection device lid.

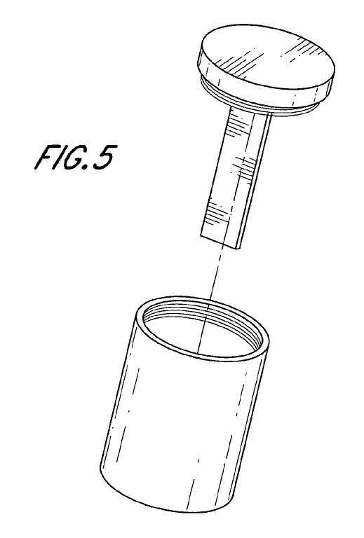

### DETAILED DESCRIPTION

In one embodiment, the invention comprises a screening test for identifying individuals at risk for breast cancer. In this embodiment the invention comprises a breast cancer risk test, as opposed to a breast cancer diagnostic test, and is thus a pre-screening test. In other words, the assay would not be used to detect breast cancer, but instead would be used to identify women who were at risk for having developed or developing breast cancer. In one preferred embodiment, a “positive” test result would therefore indicate that the subject is at risk for having developed or developing breast cancer. An individual testing “positive” could then be further screened for a malignancy or be considered for preventative measures.

Different embodiments may be directed at different general or specific risks to be assessed. One embodiment, for example, could be specifically directed at detecting a subject's higher risk for developing breast cancer. Another embodiment could be specifically directed at detecting a subject's higher risk for having developed breast cancer. Yet another embodiment would be directed at detecting subjects at higher risk for either developing or having developed breast cancer. A further embodiment could be directed at detecting specific indicators of developing or having developed breast cancer as described below.

In another embodiment of the present invention, a method of screening individuals at risk for developing or having developed breast cancer is provided. As described above, the risk assessment can be made more general or specific depending upon the particular risks desired to be detected. Subjects who test positive with the screening apparatus or method could be further screened for a malignancy or considered for preventative measures depending on the particular risk assessed.

Breast cancer begins in the lining of the ductal lobular unit. These units coalesce into lobes that emerge through the nipple in 6-8 accessible openings. In clinical studies, 80-90% of premenopausal women have been found to be able to express nipple aspirate fluid (NAF or ductal fluid, hereinafter used interchangeably) after three independent efforts. Although the volume of fluid obtained may be low, breast cancer risk markers can still be detected.

According to several embodiments of the present invention, breast ductal fluid is obtained for a Breast Fluid Test. Ductal fluid can be obtained by massaging the breast. One technique includes pressing inward on the breast, moving from the chest wall toward the nipple. This massage helps move fluid from the back of the duct toward the natural openings in nipple, where the ductal fluid is expressed.

Although breast massage to obtain ductal fluid, the most cost-effective option, is used in several embodiments, other methods for and apparatuses directed at obtaining ductal fluid can also be used. Methods for obtaining ductal fluid can include methods using aspiration, suction, rinsing and other means known to one of ordinary skill in the art. Likewise, apparatuses for obtaining ductal fluid can include apparatuses employing aspiration, suction, rinsing and other means known to one of ordinary skill in the art. However, because the objective of several embodiments of the invention is to provide a pre-screen rather than a full comprehensive screen or a diagnosis, manual massage is used in several embodiments.

In several embodiments, mechanical apparatuses for obtaining ductal fluid are not used, and only massage is instructed (in for example, instructions for use as provided in a kit). For example, in one embodiment, ductal lavage or needle aspiration are not recommended or used. In one embodiment, contrary to teachings in the art that sufficient amounts of fluid cannot be obtained with either ductal lavage, needle aspiration, or invasive procedures, several embodiments of the invention are sufficiently sensitive to detect markers in small amounts of ductal fluid obtained solely through massage. Thus, as opposed to mechanical aspirators which are used to obtain greater volumes of ductal fluid for assays which may lack sensitivity, manual massage is operable in several embodiments of the present invention. Further, smaller volumes of ductal fluid are sufficient for several embodiments of the present invention because the objective of some embodiments is to identify women who are at risk of having or developing breast cancer (e.g., a pre-screen), and not used to detect breast cancer. For systems that aim to detect breast cancer, ductal lavage, needle aspiration, and other mechanical aspirators may be necessary to obtain greater quantities of ductal fluid. Manual massage combined with a well to directly capture small volumes of ductal fluid is advantageous in several embodiments because it reduces the loss of fluid on secondary devices (such as vials, needles, aspirators, etc). Thus, in one embodiment, direct contact of ductal fluid onto wells (or other portions of a panel or support system) offers higher sample yield because an intermediate collection step is absent.

In addition to being cost-effective in many embodiments, manual breast massage is advantageous because it avoids damage or irritation to sensitive breast tissue. Thus, in addition to providing the user with a more comfortable system, biological markers may be obtained in a more undisturbed and stable state.

In one embodiment in which a secondary device is used, a suction cup constructed of a rigid or semi-rigid material is provided. Semi-rigid and rigid materials can include plastics, rubbers, glass, polymers or any other appropriate material that can be applied to or over the nipple to aid in obtaining fluid. In another embodiment, a suction cup as described above is connected to a source of suction. In a further embodiment, the connection between the suction cup and source of suction comprises a tubular device. In one embodiment, the source of suction is comprised of a hand-bulb. In another embodiment, the source of suction is comprised of a motorized or other type of pump. In another embodiment, the suction applied is provided by an individual.

In another embodiment, one or more ducts are lavaged. In one lavaging embodiment, a trained professional inserts a microcatheter into one or more of the ducts' natural openings on the nipple. In one further embodiment, suction is applied to the microcatheter to remove ductal fluid that might be present. In another further embodiment, sterile fluid is used to flush the duct and collect any breast fluid occupied within the duct. Sterile fluid can also be added to collected ductal fluid where additional fluid volume is needed and a diluted sample is acceptable. It will be understood that all described embodiments making use of ductal fluid could also make use of the diluted sample described. In some embodiments, the ductal fluid is concentrated.

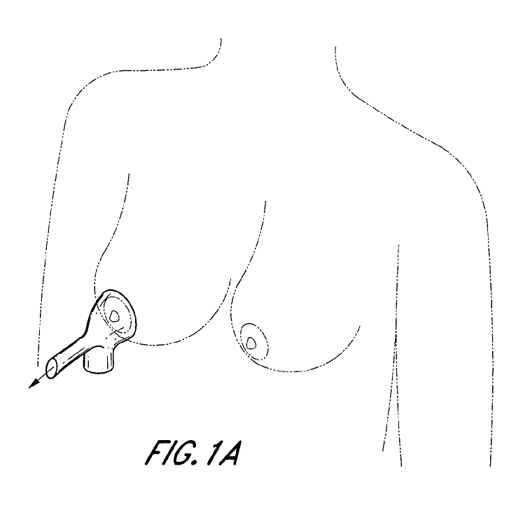

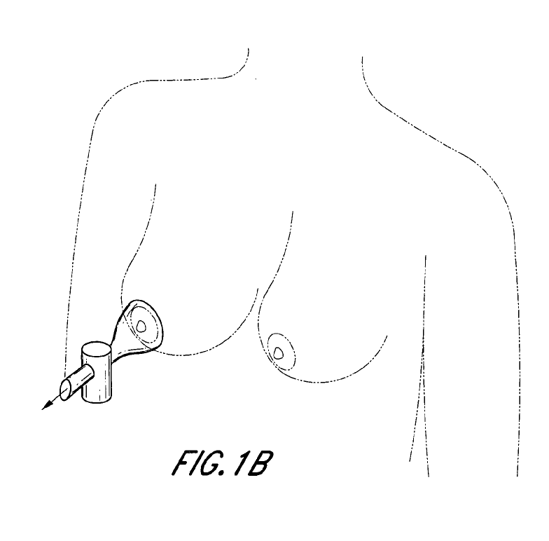

In one embodiment the suction cup also serves as a collection device. An example of such an embodiment is depicted in FIG. 1A. The arrow shows where the tubular region or a tubular device would extend to a source of suction. In another embodiment, a fluid collection device is interposed between the source of suction and the suction cup or microcatheter. One example of such an embodiment is depicted in FIG. 1B. The arrow again shows where the tubular region or a tubular device would extend to a source of suction. The collection devices can serve to collect or hold the ductal fluid obtained from the breast whether obtained by massage, aspiration, suction, lavaging or other means. The methods for obtaining ductal fluid described can be self-administered or administered by a trained individual.

In one preferred embodiment, a Breast Fluid Test comprises one or more breast cancer risk marker panels having chemical or biological breast cancer risk marker detectors (e.g., antibodies or antigens) to measure the presence or absence of certain breast cancer risk markers. Breast cancer risk markers are markers particularly selected for their association with an increased risk for developing or having developed breast cancer.

Breast cancer risk markers that can be used according to several embodiments of the invention include, but are not limited to, one or more of the following:

Basic fibroblast growth factor (bFGF): bFGF is one of the family of growth factor peptides important in wound healing and embryological development. It has been found to promote endothelial cell proliferation and angiogenesis. High levels in nipple discharge have been shown to have a significant association with breast cancer.

Apolipoprotein D (Apo D): Apo D, a high density lipoprotein, is a particle-associated protein. Apo D expression is thought to be a marker of cellular differentiation and growth arrest. Apo D is also an estrogen-inhibited protein and its levels are known to be increased in nipple aspirate fluid from women on HRT.

Mammaglobin: Mammaglobin, a cell protein, recently named as a secretoglobin has been discussed as a promising diagnostic marker in breast cancer for over 10 years. It is found in normal breast epithelial cells but is overexpressed in breast cancer.

Prostate surface antigen (PSA): PSA, or human glandular kallikrein 3 (hK3), a kallikrein-like serine protease is the most valuable tumor marker for the screening, diagnosis and management of human prostate carcinoma. Recently, it has become widely accepted that PSA is also present in many nonprostatic sources including breast secretions.

Vitamin D binding protein (VDR): Specific VDR gene polymorphisms have been shown to be associated with breast cancer risk in a United Kingdom Caucasian population. Overexpression of Vitamin D-binding protein in NAF from tumor-bearing breasts has been shown.

Gross cystic disease fluid protein (GCDFP15): GCDFP15 is a secretory protein found in various body fluids. Altered levels of this protein have been confirmed to be altered (down-regulated) in breast cancer. Significantly higher concentrations of this protein were found in the NAF of Asian women.

Alpha-lactalbumin (a-lactalbumin): A-lactalbumin is a major milk protein. One study found that an activated form of a-lactalbumin killed cancer cells. Other biological functions that have been attributed to a-lactalbumin are inhibition of epithelial cell growth, induction of apoptosis and a cell lytic activity with the assumption that cancer tissue may lose its ability to produce alpha-lactalbumin.

Cathepsin D: The Calthepsin D protein enzyme has been studied in nipple aspirate fluid and ductal lavage fluid and seen to be related to estrogen activity. Cathepsin D is present in high concentrations in NAF (>3 μg/mL) and is relatively stable over time. Cathepsin D is the product of a gene that is promoted by estradiol. Levels of cathepsin D in NAF increase in response to estrogens and decrease in response to estrogen deprivation. Cathepsin D is also present in human eccrine sweat.

Annexin I/II/V: Annexins I, II and V belong to a family of structurally related calcium and phospholipid-binding proteins implicated in signal transduction, DNA replication, cell proliferation and apoptosis. The decreased expression of Annexin I, II and V proteins has been reported in different types of cancer.

Free Her-2/neu. Free Her-2/neu has been identified in nipple aspirate fluid and thought to be associated with an increased risk of breast cancer when present and overexpressed in benign breast tissue.

Alpha-fetoprotein: Alpha-fetoprotein, a fetal growth-regulating protein, also has been found under certain conditions to inhibit the growth of breast cancer cells, according to Barbara Richardson, at Texas A&M. Alpha-fetoprotein receptors have also been noted in the cytosol of breast cancer cells.

C Reactive Protein: C reactive protein is an acute phase protein produced by the liver and is a known marker for inflammation. Although the exact role of inflammation in cancer is not well understood, some organs of the body show greater risk of cancer when they are chronically inflamed.

Carcinoembryonic antigen is a glycoprotein involved in cell adhesion. It is normally produced during fetal development, but the production of CEA typically stops before birth. Therefore, it is not usually present in the blood of healthy adults, although levels may be found in breast cancer.

Receptors for the markers described above may themselves be markers. Further, other breast cancer risk markers may be used instead of, or in addition to, the markers identified above. The breast cancer risk marker detectors, according to some embodiments, include monoclonal and polyclonal antibodies. The determination of whether to use monoclonal antibodies, polyclonal antibodies or a combination thereof depends on considerations that are well known to one of skill in the art including cost, accuracy and availability. In one embodiment, the panel includes measures of independent markers of proliferation, inflammation, hormones, and proteins. In other embodiments, the panel includes one or more markers for bacteria, viruses, fungi, yeast, and/or other microorganisms, and/or the products of such organisms. Cellular markers that can be easily measured without diagnostic cytology can also be used. Atypical cells, such as cells with altered morphologies, may be detected in another embodiment.

In one embodiment, the markers comprise PSA and Alpha-fetoprotein, together or in combination with any one of the markers identified herein. In another embodiment, the markers consist only of or only essentially of PSA and Alpha-fetoprotein. In one embodiment, the markers comprise EGF, Apolipoprotein D and urokinase, together or in combination with PSA and Alpha-fetoprotein. Other combinations, which are disclosed herein, may also be used.

In one embodiment, the actual volume or density of the fluid obtained may be used as a marker. For example, in one embodiment, excess fluid may be considered a marker which warrants further screening.

In several embodiments, the Breast Fluid Test comprises a plurality of test strips, marker panels or support members (hereinafter used interchangeably). These marker panels include immunoassay strips adapted to detect breast cancer risk markers as described above. In one embodiment, immunoassay strips are formed by compressing nonwoven fibers into a narrow strip and then coating the strips with reactive antibody or antigen marker detectors. In several embodiments incorporating test strips, the test strips are coated with one or more of the breast cancer risk marker detectors corresponding to the breast cancer risk markers disclosed above or known in the art. In several other embodiments, one or more breast cancer risk marker detectors are applied to selected regions of one or more test strips.

In one embodiment, an antibody pair is used for each target protein marker to form a sandwich immunoassay. One of the antibodies (Ab 1) is conjugated and deposited on the conjugate pad. The second antibody (Ab 2) is immobilized onto a nitrocellulose membrane as a test line. The finished test strip comprises a sample pad, gold conjugate pad, nitrocellulose membrane with test line and a control line and an absorption pad at the end of the test strip.

In one embodiment, when ductal fluid contacts or is added to the sample pad, the fluid will filter through the sample pad and migrate to the conjugate pad, dissolve the Ab 1-gold and move onto the nitrocellulose membrane by the capillary action. When the liquid mixture reaches the test line with immobilized Ab2, a sandwich of Ab1-gold-target protein-Ab2 will be formed on the test line and show as a red line in the test zone. If there is no target protein in the NAP sample, no sandwich complex will be formed and no color will be observed in the test zone. Therefore, like the commercial hCG test, a positive sample will provide a colored test line (e.g., red) and a negative sample will provide a clear test line.

In alternative embodiments, the appearance of a color would indicate a negative result. For example, red would indicate positive and green would indicate negative. In other embodiments, an intermediate or undetermined result would provide another color (e.g., yellow). In other embodiments, the strip is colored, and the removal of color would indicate a positive result. In another embodiment, a digital readout is provided that would display an indicator notifying of the presence of absence of risk. As an example, the indicator could display the words “risk” or “no risk.”

In several embodiments, the Breast Fluid Test comprises a control line. In one embodiment, the control line is prepared with rabbit-gold conjugate and an immobilized goat anti-r-IgG with the same protocol. A red line (or other control indicator) will show in the control zone to assure the proper performance of the test strip. A panel of target proteins can be detected by more than one test line on the strip and by the incorporation of more than one strip in a plastic test device.

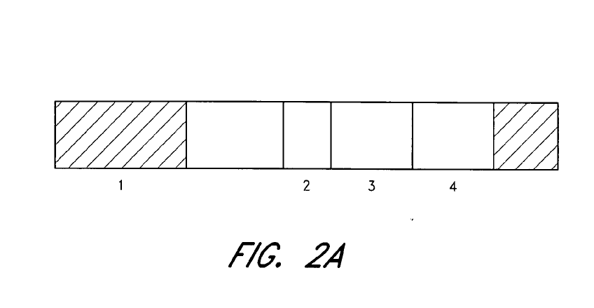

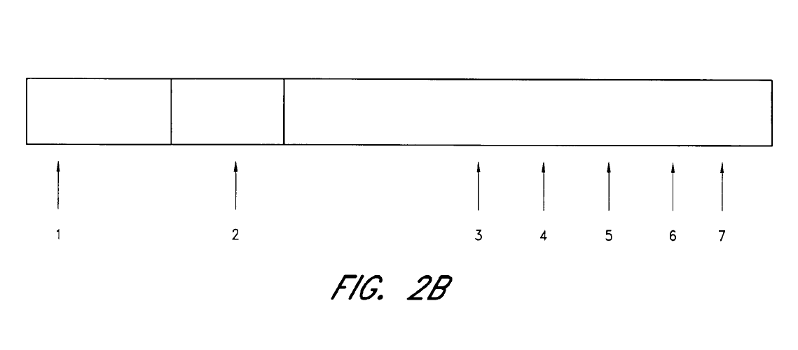

FIG. 2A shows a diagram of a two marker test strip (e.g., hemoglobin and transferrin). In this embodiment absorption end 1 captures fluid which passes over or through the marker test strip to first marker section 2. First marker section 2 contains a marker detector associated with the first marker desired to be tested for. Second marker detector section 3 contains a second marker detector associated with the second marker desired to be test for. Control line section 4 acts as an indicator to ensure proper performance of the test strip. In one embodiment control line section 4 contains a marker detector which reacts with all ductal fluid to ensure an indicator is provided when ductal fluid reaches control line section 4.

FIG. 2B shows a diagram of a five marker test. In this embodiment a handle 1 allows the test strip to be held by the individual conducting the test. A well or absorption portion 2 serves to capture ductal fluid. Five marker detectors 3-7 are placed along the remainder of the test strip in a similar manner as described above in connection with FIG. 2A.

In several embodiments, the Breast Fluid Test comprises a test strip. In one embodiment, the test strip has the following dimensions: a height in the range of about 1 mm to about 10 mm, a length in the range of about 1 cm to about 15 cm, and a thickness in the range of about 0.001 mm to 3 mm. Other dimensions may also be used.

A plurality of strips may also be used. As an example, a star-shaped embodiment as shown in illustration C may also be used. In this embodiment a central absorption portion is placed on or over the nipple. The central absorption portion captures expressed fluid which is applied to the connected test strips as described. The test strips can lead radially outward from the center portion as shown. The test strips are not required to lead radially outward or point in different directions, however. The test strips could, for example, all point down and away from the nipple to take advantage of the gravitational pull that would help to pull the ductal fluid down and over the test strips. The central absorption portion and test strips could further be combined into a single device whereby the marker detectors are applied to the interior of the collection device which would then also functions as a test strip.

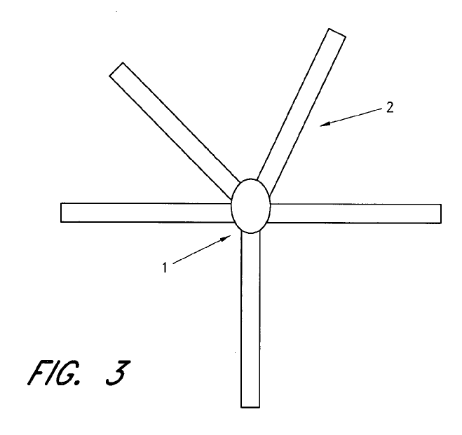

FIG. 3 shows one example of the star-shaped alternative embodiment of the Breast Fluid Test described above. This embodiment is comprised of a center well or absorption portion 1 for capturing ductal fluid. The marker panels or test strips 2, each containing at least one marker detector, can be seen leading away from the center well. A control line is optional and can be placed anywhere on the device or at the end of one or more of the test strips 2.

In one embodiment, the Breast Fluid Test comprises a marker panel that would detect the presence of one or more markers, and which is incorporated into, adhered to, attached to or otherwise coupled to a testing device that is used to capture ductal fluid. In one embodiment, the Breast Fluid Test could be self applied as a “band-aid” or patch that could collect fluid and test it simultaneously, which is particularly advantageous since for some women only about 1-10 microliters of fluid are elicited from the breast. In one embodiment, this system is adapted to assay small quantities of ductal fluid, such as 1-5 μl, 5-10 μl, 10-15 μl, 15-20 μl, 20-30 μl, 30-50 μl, and 50-500 μl. In one embodiment, the device has the following dimensions: a height in the range of about 0.5 cm to about 5 cm, a length in the range of about 1 cm to about 15 cm, and a thickness in the range of about 0.001 mm to 5 mm. Other dimensions may also be used.

The Breast Fluid Test may be rectangular, square, circular, oval, triangular, or amorphous. Different configurations and sizes for the Breast Fluid Test apparatus may also be used. Adhesive may be provided on the strip or patch to facilitate placement or securing of the device to the patient. The adhesive can be activated by moisture, or may be covered with a removable covering.

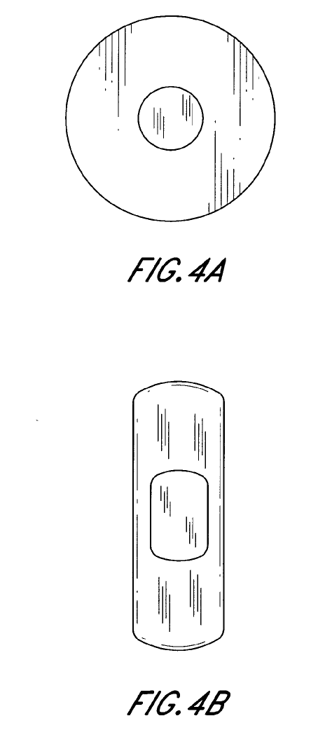

In one bandage embodiment, a test strip is placed on an adhesive backing in much the same way gauze is placed on a band-aid. The test strip portion of the bandage can then be placed directly over the nipple. The breast can then be massaged or suction can be applied over the bandage. The expressed ductal fluid is thereby applied directly to the test strip. As with band-aids, the Breast Fluid Test bandage embodiments can take on many different shapes and sizes. FIGS. 4A and 4B show two alternative bandage embodiments with the test strip portions centered on the adhesive backings.

In one embodiment, the Breast Fluid Test comprises a well or absorption portion to capture ductal fluid. In one embodiment, a single well or absorption portion is provided. In other embodiments, a plurality of wells or absorption portions are provided. The well or absorption portion can also contain one or more marker detectors. By containing marker detectors, the well or absorption portions serve to both collect and test the expressed ductal fluid.

In several embodiments, antibody or antibody fragments used in embodiments of the invention include monoclonal or polyclonal antibodies made by enzyme or chemical procedures described by Tijssen, P. Laboratory Techniques In Biochemistry And Molecular Biology: Practice And Theories Of Enzyme Immunoassays. New York: Elsevier (1985), herein incorporated by reference.

Marker detectors (such as antigen and antibodies, or fragments thereof) may be bonded to the panel (or other support member) by processes, such as those disclosed in U.S. Pat. Nos. 3,873,683, 4,003,988, 4,419,453 and Tijssen, Laboratory Techniques In Biochemistry And Molecular Biology: Practice And Theories Of Enzyme Immunoassays. Elsevier Science Publishers, (1985) pages 297-328, herein incorporated by reference.

The panel (or other support member) may comprises natural and synthetic organic and inorganic polymers. In one embodiment, the panel comprises a nylon or nitrocellulose membrane. In another embodiment, the panel comprises polystyrene, and/or polyolefins, Polymers may also include, but are not limited to, polyethylene, polypropylene, polybutylene, polyesters, polyamides, cellulose and cellulose derivatives, acrylates, methacrylates, vinyl polymers, and nylon. In some embodiments, the panel comprises silica gel, silicon wafers, glass, paper, insoluble protein, metals, and metal oxides. In other embodiments, the polymer comprises gels, lipopolysaccharides, silicates, agarose, polyacrylamides or polymers which form aqueous phases such as dextrans, polyalkylene glycols, or surfactants, e.g. amphophilic compounds such as phospholipids, long chain (12-24 carbon atoms) alkyl ammonium salts, etc. Combination of the materials identified above may also be used in several embodiments.

In one embodiment, the Breast Fluid Test comprises one or more filters portion. In one embodiment, the filter will be applied directly over the nipple in order to effectively transmit the ductal fluid to the marker detectors. In another embodiment the marker detectors are applied directly to the filter. In some embodiments, the expressed ductal fluid is applied to the filter which would show a color change (or other indication when wet). As with all embodiments described herein, one or more of the previously described methods (e.g., massaging, aspiration, suction, or lavaging) can be used to obtain the ductal fluid which is applied to the Filter.

In one embodiment, the breast cancer risk marker panel or test strip is incorporated into a dipstick-like test that could be used to analyze expressed ductal fluid. In one such embodiment, for example, the ductal fluid would be obtained by one of the methods described above, submersing the test strip, entirely or partially, in the expressed ductal fluid. In one embodiment, the ductal fluid is collected using one of the described methods and held in a collection device. The test strip is then submersed, to the extent possible, in the collected ductal fluid. In another embodiment, the test strip is included in the collection device and is submersed as the fluid is accumulated in the collection device. In a further embodiment, an example of which is depicted in FIG. 6, the marker panel is incorporated into, adhered to, attached to, or otherwise coupled with a lid or cover for a fluid collection device. The lid or cover for the fluid collection device is connected to the fluid collection device to create a fluid-tight seal. The fluid collection device can then be rotated or shaken to ensure the available ductal fluid is absorbed by the marker panel.

In other embodiments, the collected fluid is applied to a breast cancer risk marker panel. In one such embodiment, for example, the ductal fluid can be poured over the marker panel. The marker panel may contain markings to indicate where the test subject or test administrator is to deposit the ductal fluid. In one embodiment, a medicine dropper, pipette or similar device is provided so that the collected fluid can be removed from the fluid collection device and placed on the marker panel with adequate specificity.

In one embodiment, the Breast Fluid Test comprises one or more test strips, wherein each test strip is specific for a single breast cancer risk marker. Accordingly, the presence of a specific marker can be determined. In one embodiment, the detection of a single marker will give a positive response. In another embodiment, all of the markers are commingled on a single strip. In this embodiment, a positive reading would only indicate that at least one of several markers was present. In still another embodiment, test strips different test strips contain different combinations of one or more markers.

In one embodiment, marker detectors for breast cancer risk markers present in all of the ducts are assayed. This embodiment is particularly advantageous in instances where fluid may not be obtained from every duct. In other embodiments, however, detectors for markers that are not necessarily present in more than one duct are used.

In one embodiment, the Breast Fluid Test would show a preference for providing a higher false positive rate, as opposed to a higher false negative rate. Because the Breast Fluid Test is designed, in one embodiment, to pre-screen individuals (or identify individuals for further screening) rather than to conduct a comprehensive screen or diagnosis, the test would tolerate a higher false positive rate than would be acceptable for a breast cancer diagnostic test (or other type of screening or diagnostic test) because it would be used as an initial screen (or pre-screen) to determine which individuals needed further workup. In some embodiments, the false positive rate is greater than 5%, 10%, 20% or 50%.

In one embodiment, the Breast Fluid Test is designed to identify all individuals that test positive for at least one marker, and is thus more sensitive than specific. In another embodiment, specificity is increased by using an assay that only indicates “positive” when two or more, three or more, four or more, or five or more markers are present in the ductal fluid. In yet another embodiment, the assay indicates “positive” only when all included markers are present.

Because of the heterogeneity of cancer, one embodiment of the Breast Fluid Test comprises a panel of multiple markers that are used to achieve the needed sensitivity and specificity of breast cancer risk determination. In one embodiment, one to five markers are used. In another embodiment, five to ten markers are used. In yet another embodiment, more than ten markers are used. For cost-effectiveness, panels of less than about six markers are used in some preferred embodiments. In one embodiment, a profile will comprise five markers, each representing different molecular pathways for cancer development.

In one embodiment, one or more of the constituents of ductal fluid are measured, as identified on Table 1. This table originally appeared in King B L & Love S M, The Intraductal Approach to the Breast: Raise D'Etre, Breast Cancer Research 2006, 8:206, which describes the significance of these ductal fluid components. The entirety of that article and description are incorporated herein by reference.

In some embodiments, abnormality of the constituent, as described in King B L & Love S M, is detected. In other embodiments, the measurement of the constituent is used as a control.

TABLE 1
Partial listing of nipple aspirate fluid constituents

| Proteins | Immunoglobulins | Fats | Hormones | Electrolytes | Cells |
|---|---|---|---|---|---|
| Alpha 1 lipoprotein | IgA | Lauric | Prolactin | Sodium | Epithelial |
| Alpha 1 acid glycoprotein | IgM | Myristic | Estrone | Potassium | Myoepithelial |
| Alpha 2 macroglobulin | IgG H chain | Myristoleic | Estradeiol | Chloride | Macrophages |
| Alpha 2 HS glycoprotein | IgG L chain | Palmitic | DHEAS | Calcium | Neutrophils |
| Alpha 1 antitrypsin | IgE | Palmitoleic | Progesterone | Phosphate | Lymphocytes |
| Trypsin | IgD | Cholesterol | Growth hormone | | Mast Cells |
| Beta liprotein | | Cholesterol epoxides | Testosterone | | Erythrocytes |
| Beta glycoprotein III | | | TGF-α | | |
| Ceruloplasmin | | | EGF | | |
| Prealbumin | | | | | |
| Human lysozyme | | | | | |
| α-Lactalbumin | | | | | |

DHEAS, dihydroepiandrosterone sulfate;
EFG, epidermal growth factor;
TGF-α, transforming growth factor-alpha.

In an alternative embodiment, the quantity of ductal fluid will be ascertained by the Breast Fluid Test. Because the presence and/or amount of ductal fluid that is elicited from the breast can itself be a risk factor for breast cancer, the Breast Fluid Test, in some embodiments, will give a positive reading upon detecting the presence or absence of ductal fluid (or a certain quantity of ductal fluid), regardless of the presence of any markers. In several embodiments, a positive indication is provided when a volume of ductal fluid greater than a predetermined volume is detected (e.g., greater than 0, 2, 4, 6, 8, or 10 microliters). In another embodiment, a positive indication is provided when greater than 20, 40, or 60 microliters is detected. In further embodiments, the Breast Fluid Test will show positive only when a certain quantity of ductal fluid and the presence of at least one marker is present. In one embodiment, a positive result would occur when a either a certain (higher) volume of ductal fluid is detected or when a certain (lower) volume is detected and a marker is detected. Thus, both presence of fluid or markers, and combinations thereof, may be used as an indicator of risk. A positive test would indicate that the user needed further workup either through cytological analysis of fluid or imaging.

Thus, in one embodiment, the invention comprises a system for detecting individuals at risk for having breast cancer, but who do not necessarily have breast cancer. In one embodiment, about 10-20% of individuals who screen positive will have breast cancer or be at high risk of developing breast cancer. In other embodiments, the rate is less than 10% or between about 20-50%. In some embodiments, the screening test provides rates greater than 50%. However, in several embodiments, the invention is not intended to diagnose breast cancer, but instead identify those individuals who should seek further testing. Likewise, in one embodiment, the invention is not intended to determine which individuals are at high risk for breast cancer, but instead identify those individuals who are at risk for falling into the high risk category.

In several embodiments, the invention can determine a range of risk. For example, in one embodiment, risk is correlated with the number of markers detected in the ductal fluid. The more markers detected, the higher the risk for wither developing breast cancer or having breast cancer. In another embodiment, the detection of a marker (e.g., a protective marker) may be reversely correlated with risk. Protective markers include, but are not limited to, tumor suppressor proteins.

In several embodiments, the marker combination used, comprising one or more markers disclosed herein, will be subject to future validation. For example, in one embodiment, subjects who test “positive” will be further screened for a malignancy. This further screening can comprise, for example, breast density testing. Breast density testing is believed to be a validated test for determining breast cancer risk. The results obtained from the further screening can be compared with different marker combinations used in the Breast Fluid Test. The validation of the marker combination can also allow the Breast Fluid Test to be specifically tailored to the group being tested. Thus, in instances where ethnicity, race, age or other distinguishing population factors can be linked to different preferred marker combinations, validation of the marker combinations can allow the Breast Fluid Test to reflect this preference.

In several embodiments, the Breast Fluid Test will be accompanied by instructions for use in a kit format. The instructions, in one embodiment, will provide information regarding proper use of the device and proper interpretation of the results. Instruction included with embodiments making use of color coding, for example, will “match” the color coding with instructions for the user. For example, the instruction may state: “if you see a red color, you may be at risk for breast cancer and require further screening.”

While this invention has been particularly shown and described with references to preferred embodiments thereof, it will be understood by those skilled in the art that various changes in form and details may be made therein without departing from the scope of the invention encompassed by the appended claims.

### REFERENCES

The following references are incorporated by reference in their entirety.
Anderson, B O et al (2007) The Breast Health Global Initiative: A Framework for Improving Breast Health Care in Limited-Resource Countries Breast Diseases Vol 17 #4 314-317
Yang, Ling et al 2005 Estimates of Cancer Incidence in China for 2000 and Projections for 2005 Cancer Epidemiol Biomarkers Prey; 14 (1) 243-250
Thomas D B et al 2002 Randomized trial of breast self-examination in Shanghai: final results, J Natl Cancer Institute 2:94(19):1445-57
Fabian, C J et all 2005 Breast-tissue sampling for risk assessment and prevention Endocrine-Related Cancer 12 185-213
Gail M H et al 1989 Projecting individualized probabilities of developing breast cancer for white females who are being examined annually J Natl Cancer Inst 81(24): 1879-1886
Wellings Sr & Jensen H M 1973 On the origin and progression of ductal carcinoma in the human breast. Journal of the National Cancer Institute 50 1111-1118
Bushnaq, Z et al 2007 Patient Variables that predict atypical cytology by Nipple duct Lavage Cancer Volume 109 #7 1247-1254
Wrensch M R et al 2001 Breast cancer risk in women with abnormal cytology in nipple aspirates of breast fluid Journal of the National Cancer Institute 93 1791-1798
Buehring G C et al 2006 Presence of epithelial cells in nipple aspirate fluid is associated with subsequent breast cancer: a 25-year prospective study Breast Cancer Research and Treatment 98: 63-70
Baltzell, K A Abnormal Cytology in NAF and Subsequent Breast Cancer Risk Presentation at the 5^{th} International Symposium on the Intraductal Approach to Breast Cancer 2007
Busbnaq, Z et al 2007 Patient Variables that predict atypical cytology by Nipple duct Lavage Cancer Volume 109 #7 1247-1254
Khan, S A et al 2004 Ductal lavage findings in women with known breast cancer undergoing mastectomy Journal of the National Cancer Institute 96 1510-1517
Petrakis, N L et al 1998 Birthplace and Yield of Nipple Aspirate Fluid in Chinese Women Cancer Epidemiology, biomarkers & Prevention 7 835-839
Sauter E R et al 2007 Biological markers of breast cancer in nipple aspirate fluid and nipple discharge are associated with clinical findings Cancer Detection and Prevention 31:1 50-58
Hemstreet G P, Rao J Y, Hurst R E, et al. Intermediate endpoint markers for chemoprevention. J. Cell. Bioc. (Suppl.), 161:93-110, 1992.
Kelloff, J O, et al, Progress in chemoprevention drug development: the promise of molecular biomarkers for prevention of intraepithelial neoplasia and cancer—a plan to move forward. Clin Cancer Res. 2006 Jun. 15; 12(12):3661-97.
Yang, Ling et al 2005 Estimates of Cancer Incidence in China for 2000 and Projections for 2005 Cancer Epidemiol Biomarkers Prey; 14 (1) 243-250
Yang, Ling et al 2005 Estimates of Cancer Incidence in China for 2000 and Projections for 2005 Cancer Epidemiol Biomarkers Prey; 14 (1) 243-250
Woo P P S et al What is the Most Cost-Effective Population-Based Cancer Screening Program for Chinese Women? Journal of Clinical Oncology 25 #6 617-624.
Jakes, R W et al 2002 Mammographic Parenchymal Patterns and Self-reported Soy Intake in Singapore Chinese Women. Cancer Epi Biomarkers & Prev 11, 608-613
Ursin, G et al 2003 Mammographic density and breast cancer in three ethnic groups. Cancer Epidemiol Biomarkers Prev.; 12(4):332-8.
Buehring G C et al 2006 Presence of epithelial cells in nipple aspirate fluid is associated with subsequent breast cancer: a 25-year prospective study Breast Cancer Research and Treatment 98: 63-70
Shepherd, J A, Herve L, Landau J, Fan B, Kerlikowske K, Cummings S R 2005 Novel use of Single X-Ray Absorptiometry for Measuring Breast Density. Technol Cancer Res Treat 4(2):173-82.

1-18. (canceled)

19. A device for identifying individuals at risk for breast cancer, the device comprising:
an adhesive configured to attach to human tissue;
a well for capturing ductal fluid from a breast duct;
at least one panel configured to contact ductal fluid captured in the well,
wherein said panel comprises at least one marker detector adapted to detect at least one marker, said at least one marker being associated with breast cancer risk;
wherein the at least one marker comprises EGF and urokinase;
wherein said marker detector is adapted to bind to the at least one marker; and
wherein said device indicates a positive response upon binding of the at least one marker.

20. The device of claim 19, wherein the panel additionally comprises transferrin.

21. The device of claim 19, further comprising:
a first antibody conjugated with gold to form a conjugate, wherein said conjugate is deposited on the panel, and
a second antibody immobilized onto a nitrocellulose membrane,
wherein captured ductal fluid at least partially dissolves the conjugate to form a liquid mixture,
wherein at least a portion of the liquid mixture is adapted to move onto the nitrocellulose membrane by capillary action and contact the second antibody,
wherein a sandwich of the conjugate and the second antibody will form if the at least one marker is present in the ductal fluid, and
wherein a color will form upon formation of the sandwich.

22. The device of claim 19, wherein the device comprises at least two panels, wherein each panel comprises a marker detector.

23. The device of claim 19, wherein the device comprises a single panel, wherein said panel comprises at least one marker detector.

24. The device of claim 19, wherein a positive response is indicated by a color change.

25. The device of claim 19, wherein the well is adapted to draw fluid from the breast duct.

26. The device of claim 19, further comprising a portion to facilitate transport of the captured fluid to the marker detectors.

27. The device of claim 19, further comprising a detector to detect the presence of ductal fluid.

28. The device of claim 19, further comprising a detector to detect a volume of captured ductal fluid.

29. The device of claim 19, further comprising an indicator to indicate the presence of ductal fluid or to indicate a volume of captured ductal fluid.

30. The device of claim 29, wherein said indicator comprises a color change.

31. A kit comprising the device of claim 19 and corresponding instructions for use.

32. A kit comprising the device of claim 19 and an aspirator for facilitating capture of ductal fluid.

33. A device for identifying individuals at risk for breast cancer, the device comprising:
an adhesive configured to attach to human tissue;
a well for capturing ductal fluid from a breast duct;
at least one panel,
wherein said panel is configured to contact ductal fluid captured in the well,
wherein said panel comprises at least one marker detector;
wherein the marker detector comprises an antibody or antigen;
wherein said antibody or antigen detects a marker selected from the group consisting of one or more of the following markers: EGF, urokinase, bFGF, A-lipoprotein D, mammaglobin, prostate surface antigen, Vitamin D binding protein, GCDFP15, alpha-Lactoalbumin, Cathepsin D, Annexin I/II/V, Free Her-2, Alpha-fetoprotein, C Reactive Protein, and Carcinoembryonic antigen;
wherein said marker detector is adapted to bind to at least one of said markers;
wherein said markers are associated with breast cancer risk; and
wherein said device indicates a positive response upon detection of at least one of said markers.

34. The device of claim 33, wherein the panel further comprises transferrin.

35. A method for identifying individuals at risk for breast cancer, the method comprising:
providing a bandage comprising an adhesive, a well and a panel;
capturing ductal fluid from a breast duct in said well;
contacting the panel with the captured ductal fluid in the well;
wherein the panel comprises at least one marker detector,
wherein said marker detector is adapted to detect markers associated with breast cancer risk; and
wherein the marker comprises at least one of EGF and urokinase; and
indicating a positive response upon detection of said marker.

36. The method of claim 35, further comprising detecting the presence of ductal fluid.

37. A system for identifying individuals at risk for breast cancer, the system comprising:
a well for capturing ductal fluid from a breast duct, wherein said ductal fluid may comprise one or more markers associated with breast cancer risk;
at least one panel adapted to contact ductal fluid captured by the well;
wherein the at least one panel comprises at least one marker detector;
wherein said at least one marker detector is adapted to detect one or more of said markers associated with breast cancer risk;
wherein said markers comprise EGF and urokinase; and
wherein said system indicates a positive response upon detection of at least one of said markers.

38. The system of claim 37, wherein said at least one panel further comprises transferrin.
